(19) United States
(12) Patent Application Publication
(10) Pub. No.: US 2026/0048225 A1
(43) Pub. Date: Feb. 19, 2026

(54) PROVIDING INTERMITTENT HYPOXIA WITH SEQUENTIAL GAS DELIVERY

(71) Applicant: THORNHILL SCIENTIFIC INC., North York (CA)

(72) Inventors: Olivia SOBCZYK, Etobicoke (CA); James DUFFIN, Toronto (CA); Ece Su SAYIN, Windsor (CA); Bryan Drew MILLER, Richmond Hill (CA); Rafay KHAN, Markham (CA); Joseph Arnold FISHER, Thornhill (CA)

(21) Appl. No.: 19/107,649

(22) PCT Filed: Sep. 6, 2023
(86) PCT No.: PCT/IB2023/058827

§ 371 Date: Feb. 28, 2025

Related U.S. Application Data
(60) Provisional application No. 63/404,505, filed on Sep. 7, 2022.

(51) Int. Cl. A61M 16/10; A61M 16/00; A61M 16/06; A61M 16/12

(52) U.S. Cl. CPC A61M 16/1005; A61M 16/0003; A61M 16/024; A61M 16/06; A61M 16/12; A61M 2016/0027; A61M 2016/1025; A61M 2016/103; A61M 2202/0208; A61M 2202/0225; A61M 2205/502; A61M 2230/205; A61M 2230/432

(57) ABSTRACT

A system (100) and a method (200) for providing intermittent hypoxia using sequential gas delivery to a subject are disclosed. The method (200) comprises the step (204) of inducing a normoxic end tidal partial pressure of oxygen using a sequential gas delivery system within a first specific number of breaths for a first duration and the step (208) of inducing a hypoxic end tidal partial pressure of oxygen using the sequential gas delivery system within a second number of breaths for a second duration and repeating the steps for a target number of cycles until a therapeutically effective dose of intermittent hypoxia is attained. The sequential gas delivery system (100) may control the end tidal partial pressure of carbon dioxide simultaneously and independently of the end tidal partial pressure of oxygen. The method (200) may be applied in the treatment of a pathological condition in a subject or for improving a health condition of a subject.

### CROSS-REFERENCE TO RELATED APPLICATION(S)

This application claims the benefit of U.S. Provisional Patent Application No. 63/404,505 entitled “USE OF SEQUENTIAL GAS DELIVERY TO DEVELOP AND ADMINISTER INTERMITTENT HYPOXIA AS THERAPEUTIC MODALITY”, filed Sep. 7, 2022, the entire contents of which are incorporated herein by reference.

### FIELD

The present specification is directed to providing intermittent hypoxia.

### BACKGROUND

Intermittent hypoxia (IH) offers a myriad of health benefits. By inducing the expression of the hypoxia-inducible factor (HIF), IH triggers a cascade of positive effects across the body including the immune system, nervous system, cardiovascular system, and skeletal system. It improves physiological functions in patients with lung conditions like chronic obstructive pulmonary disease (COPD), emphysema, and asthma, and sustains breathing in amyotrophic lateral sclerosis (ALS). IH enhances cognitive function in the elderly, reduces depression, and improves quality of life in heart-related ailments. It regulates blood pressure, lipids, and immune responses while countering chronic inflammation.

Current practices for providing intermittent hypoxia to subjects use rebreathing circuits or non-rebreathing masks such as the Everest Summit II™ (Hypoxico Inc., New York, United States) which rely on trial and error to target and maintain hypoxia. Furthermore, the actual arterial concentration of oxygen is unknown and not repeatable.

### SUMMARY

An aspect of the disclosure provides a method of treating a pathological condition in a subject. The method includes (a) inducing a normoxic end tidal concentration of oxygen in the subject within a first number of breaths using a sequential gas delivery system and maintaining the normoxic end tidal concentration of oxygen in the subject for a first duration of time; (b) inducing a hypoxic end tidal concentration of oxygen in the subject within a second number of breaths using the sequential gas delivery system and maintaining the hypoxic end tidal concentration of oxygen in the subject for a second duration of time, and (c) repeating steps (a) and (b) for a target number of cycles to attain a therapeutically effective dose.

The first number of breaths may be one. The second number of breaths may be one.

The pathological condition is one of chronic obstructive lung disease, emphysema, bronchitis, asthma, spinal cord injury, Alzheimer's disease, dementia, depression, myocardial ischemia, angina, myocardial infarction, coronary artery disease, heart failure, hypertension, metabolic syndrome, inflammatory disease, and lung disease.

The method may include the step of maintaining an end tidal concentration of carbon dioxide in the subject using the sequential gas delivery system while performing steps (a) to (c). The method may include the steps of: inducing a first end tidal concentration of carbon dioxide in the subject using the sequential gas delivery system and maintaining the first end tidal concentration of carbon dioxide while inducing the normoxic end tidal concentration of oxygen, and inducing a second end tidal concentration of carbon dioxide in the subject using the sequential gas delivery system and maintaining the second end tidal concentration of carbon dioxide while inducing the hypoxic end tidal concentration of oxygen. The first end-tidal concentration of carbon dioxide may be selected to induce normocapnia, hypocapnia, or hypercapnia. The second end-tidal concentration of carbon dioxide may be selected to induce normocapnia, hypocapnia, or hypercapnia.

The method may include the step of inducing a normoxic end tidal concentration of oxygen and a normocapnic end tidal concentration of carbon dioxide for a rest period and repeating steps (a) to (c) after the rest period. Performance of steps (a) to (c) may constitute a set, and the method may include the step of repeating the set for a pre-determined number of sets. The method may include the step of repeating the set for a predetermined number of times in a day and for a predetermined number of days.

Another aspect of the disclosure provides a method of improving a health condition of a subject. The method includes (a) inducing a normoxic end tidal concentration of oxygen within a first number of breaths using a sequential gas delivery system and maintaining the normoxic end tidal concentration of oxygen for a first duration of time; (b) inducing a hypoxic end tidal concentration of oxygen within a second number of breaths using the sequential gas delivery system and maintaining the hypoxic end tidal concentration of oxygen for a second duration of time, and (c) repeating steps (a) and (b) for a target number of cycles to attain a therapeutically effective dose.

The health condition is one of exercise tolerance, altitude tolerance, aerobic capacity, and athletic endurance.

The method may include the step of maintaining an end tidal concentration of carbon dioxide in the subject using the sequential gas delivery system while performing steps (a) to (c). The method may include the steps of: inducing a first end tidal concentration of carbon dioxide in the subject using the sequential gas delivery system and maintaining the first end tidal concentration of carbon dioxide while maintaining the normoxic end tidal concentration of oxygen, and inducing a second end tidal concentration of carbon dioxide in the subject using the sequential gas delivery system and maintaining the second end tidal concentration of carbon dioxide while inducing the hypoxic end tidal concentration of oxygen. The first end tidal concentration of carbon dioxide may be the same as the second end tidal concentration of carbon dioxide. The first end-tidal concentration of carbon dioxide may be selected to induce normocapnia, hypocapnia, or hypercapnia. The second end-tidal concentration of carbon dioxide may be selected to induce normocapnia, hypocapnia, or hypercapnia. Using the sequential gas delivery system, the end tidal concentration of carbon dioxide can be maintained independently of the extent and pattern of the subject's breathing and independently of the end tidal concentration of oxygen.

The method may include inducing a normoxic end tidal concentration of oxygen and a normocapnic end tidal concentration of carbon dioxide for a rest period and repeating steps (a) to (c) after the rest period. Performance of steps (a) to (c) may constitute a set, and the method may include the step of repeating the set for a pre-determined number of sets. The method may include the step of repeating the set for a predetermined number of times in a day and for a predetermined number of days.

A further aspect of the disclosure provides the use of a sequential gas delivery system in the treatment of a pathological condition.

A further aspect of the disclosure provides the use of a sequential gas delivery system in the enhancement of a health condition.

A further aspect of the disclosure provides the use of a sequential gas delivery system to precondition an organ for transplantation.

A further aspect of the disclosure provides the use of a sequential gas delivery system to precondition an organ or tissue prior to surgery during which the blood supply to these organs and tissues is likely to be interrupted.

A further aspect of the disclosure provides the use of a sequential gas delivery system to improve bone remodelling.

A further aspect of the disclosure provides the use of a sequential gas delivery system to stimulate erythropoietin production in a subject.

A further aspect of the disclosure provides a method of determining a therapeutically effective dose of hypoxia. The method also includes (a) assessing a baseline condition for a population of subjects; (b) providing intermittent hypoxia to the population of subjects using a sequential gas delivery system, (d) assessing a post-treatment condition for the population of subjects, (e) computing the difference between the baseline condition and the post-treatment condition for the population of subjects, (f) comparing the computed differences within the population of subjects, and (g) determining a therapeutically effective dose of intermittent hypoxia based on the comparison.

In some embodiments of the method, the population of test subjects includes a test group and a control group and providing intermittent hypoxia to the population of subjects may include the steps of providing a test dose of intermittent hypoxia to the test group; and providing a control dose of intermittent hypoxia to the control group. The test and control doses of intermittent hypoxia are characterized by at least one of a partial pressure of end tidal oxygen induced during normoxia, a partial pressure of end tidal oxygen induced during hypoxia, a partial pressure of a carbon dioxide induced during normoxia, a partial pressure of a carbon dioxide induced during hypoxia, a first duration, a second duration, a target number of cycles, a rest period, and a total number of sets, a target number of sets per day, and a target number of days. Comparing the computed differences within the population of subjects may include the step of comparing the computed differences for the test group to the computed differences for the control group. The population of subjects is selected to share a common attribute. The common attribute is one of a health condition, pathological condition, sex, age, race, body weight, and height. The common attribute is one of chronic obstructive lung disease, emphysema, bronchitis, asthma, spinal cord injury, Alzheimer's disease, dementia, depression, myocardial ischemia, angina, myocardial infarction, coronary artery disease, heart failure, hypertension, metabolic syndrome, inflammatory disease, and lung disease.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a block diagram of a system for providing intermittent hypoxia.

FIG. 2 is a flowchart of a method for providing intermittent hypoxia.

FIG. 3 is a graph showing exemplary performance of one embodiment of the method of FIG. 2.

FIG. 4 is a flowchart showing exemplary performance of another embodiment of the method of FIG. 2.

FIG. 5 is a graph showing exemplary performance of one embodiment of the method of FIG. 4.

FIG. 6 is a flowchart showing exemplary performance of another embodiment of the method of FIG. 2.

FIG. 7 is a flowchart of a method for determining a therapeutically effective dose of hypoxia.

### DETAILED DESCRIPTION

### List of Abbreviations

The following abbreviations are used herein:

| | |
|---|---|
| ALS | amyotrophic lateral sclerosis |
| ASIC | application-specific integrated circuit |
| CO₂ | carbon dioxide |
| CPU | central processing unit |
| COPD | chronic obstructive pulmonary disease |
| EEPROM | electrically erasable programmable read-only memory |
| FPGA | field-programmable gate array |
| IH | intermittent hypoxia |
| N₂ | nitrogen |
| O₂ | oxygen |
| PₐCO₂ | arterial partial pressure of carbon dioxide |
| PₐO₂ | arterial partial pressure of oxygen |
| P_{ET}CO₂ | end tidal partial pressure of carbon dioxide |
| P_{ET}O₂ | end tidal partial pressure of oxygen |
| PCO₂ | partial pressure of carbon dioxide |
| PO₂ | partial pressure of oxygen |
| RAM | random access memory |
| ROM | ready-only memory |
| SGD | sequential gas delivery |

### Definitions

“About” herein refers to a range of ±20% of the numerical value that follows. In one example, the term “about” refers to a range of ±10% of the numerical value that follows. In one example, the term “about” refers to a range of ±5% of the numerical value that follows.

“Dose” herein refers to the amount and frequency of hypoxia that is provided to a subject during a treatment of intermittent hypoxia. Dose includes but is not limited to the P_{ET}O₂ induced during hypoxia, the duration of hypoxia, the P_{ET}O₂ induced during normoxia, the duration of normoxia, the first P_{ET}CO₂, the second P_{ET}CO₂, the number of cycles, the rest period, the total number of sets, the number of sets per day, the number of days, or a combination thereof.

“Health condition” herein refers to an anatomical, physiological, or mental state of a subject.

“Hypercapnic” herein refers to blood with abnormally high CO₂ levels. Generally, a hypercapnic PₐCO₂ is above about 45 mmHg.

“Hyperoxic” herein refers to blood with abnormally high O₂ levels. Generally, a hyperoxic PₐO₂ is above about 100 mmHg.

“Hypocapnic” herein refers to blood with abnormally low CO₂ levels. Generally, a hypocapnic PₐCO₂ is below about 35 mmHg.

“Hypoxic” herein refers to blood with abnormally low O₂ levels. Generally, a hypoxic PₐO₂ is below about 80 mmHg.

“Normocapnic” herein refers to blood with normal CO₂ levels. Generally, a normocapnic PₐCO₂ is between about 30 mmHg and about 50 mmHg.

“Normoxic” herein refers to blood with normal O₂ levels. Generally, a normoxic PₐO₂ is between about 70 mmHg and about 110 mmHg.

“Pathological condition” herein refers to an abnormal anatomical, physiological, or mental manifestation of a disease in a subject.

“Therapeutically effective dose” herein refers to the minimum dose of intermittent hypoxia that yields a therapeutic benefit to a subject.

### System and Methods

The present disclosure provides an improved system and method for providing intermittent hypoxia to a subject.

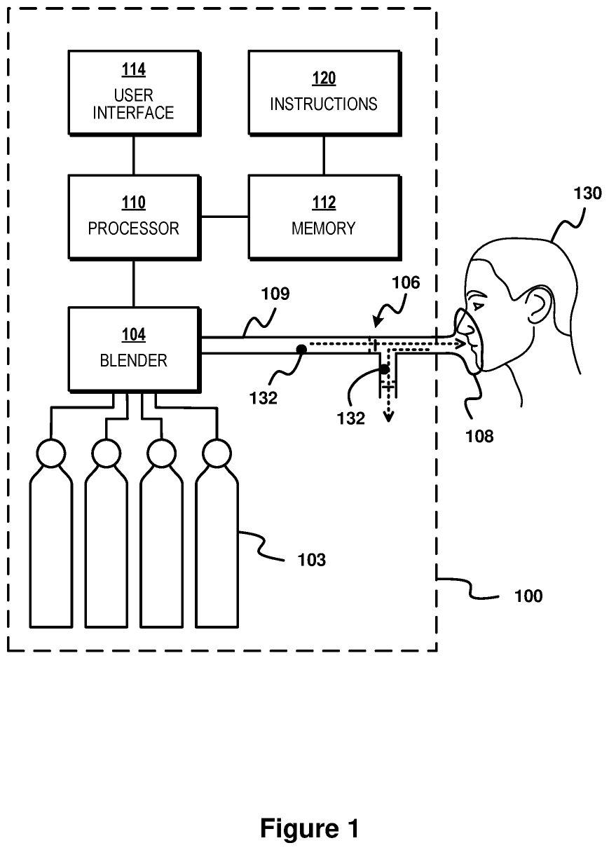

FIG. 1 is a block diagram of a system for providing intermittent hypoxia to a subject.

The system 100 comprises a sequential gas delivery (SGD) system to provide sequential gas delivery to a subject 130. The system 100 is configured to target an end tidal partial pressure of oxygen (P_{ET}O₂). The system 100 includes gas supplies 103, a gas blender 104, a mask 108, a processor 110, and memory 112. In some implementations, the system 100 includes a user interface 114. The system 100 is configured to control the P_{ET}O₂ and end tidal partial pressure of carbon dioxide (P_{ET}CO₂) by generating predictions of gas flows to actuate target end-tidal values. Using the system 100, the P_{ET}O₂ can be targeted independently of the extent and pattern of the subject's breath. Furthermore, the P_{ET}O₂ can be targeted independently of the targeted P_{ET}CO₂.

In particular implementations, the system 100 is a RespirAct™ (Thornhill Medical™, Toronto, Canada). For further information regarding sequential gas delivery, U.S. Pat. No. 8,844,528, US Publication No. 2018/0043117, and U.S. Pat. No. 10,850,052, which are incorporated herein by reference, may be consulted.

The gas supplies 103 may provide carbon dioxide (CO₂), oxygen (O₂), nitrogen (N₂), and air, for example, at controllable rates, as defined by the processor 110. A non-limiting example of the gas mixtures provided in the gas supplies 103 is:
Gas A: 10% O₂, 90% N₂;
Gas B: 10% O₂, 90% CO₂;
Gas C: 100% O₂; and
Calibration gas: 10% O₂, 9% CO₂, 81% N₂.

The gas blender 104 is connected to the gas supplies 103, receives gases from the gas supplies 103, and blends received gases as controlled by the processor 110 to obtain a gas mixture, such as a first gas (G1) and a second gas (G2) for sequential gas delivery.

The second gas (G2) is a neutral gas in the sense that it has about the same partial pressure of carbon dioxide (PCO₂) as the gas exhaled by the subject 130, which includes about 4% to 5% carbon dioxide. In some examples, the second gas (G2) may include gas actually exhaled by the subject 130. The first gas (G1) has a partial pressure of oxygen (PO₂) that is equal to the target P_{ET}O₂ and preferably no significant amount of carbon dioxide. For example, the first gas (G1) may be air (which typically has about 0.04% carbon dioxide), may consist of 21% oxygen and 79% nitrogen, or may be a gas of similar composition, preferably without any appreciable CO₂.

The processor 110 may control the gas blender 104, such as by electronic valves, to deliver the gas mixture in a controlled manner.

The mask 108 is connected to the gas blender 104 and delivers gas to the subject 130. The mask 108 may be sealed to the subject's face to ensure that the subject only inhales gas provided by the gas blender 104 to the mask 108. In some examples, the mask is sealed to the subject's face with skin tape such as Tegaderm™ (3M, Saint Paul, Minnesota). A valve arrangement 106 may be provided to the system 100 to limit the subject's inhalation to gas provided by the gas blender 104 and limit exhalation to the room. In the example shown, the valve arrangement 106 includes an inspiratory one-way valve from the gas blender 104 to the mask 108, a branch between the inspiratory one-way valve and the mask 108, and an expiratory one-way valve at the branch. Hence, the subject 130 inhales gas from the gas blender 104 and exhales gas to the room.

The gas supplies 103, gas blender 104, and mask 108 may be physically connectable by a conduit 109, such as tubing, to convey gas. One or more sensors 132 may be positioned at the gas blender 104, mask 108, and/or conduits 109 to sense gas flow rate, pressure, temperature, and/or similar properties and provide this information to the processor 110. Gas properties may be sensed at any suitable location, so as to measure the properties of gas inhaled and/or exhaled by the subject 130.

The processor 110 may include a central processing unit (CPU), a microcontroller, a microprocessor, a processing core, a field-programmable gate array (FPGA), an application-specific integrated circuit (ASIC), or a similar device capable of executing instructions. The processor 110 may be connected to and cooperate with the memory 112 that stores instructions and data. The term “processor” as used herein may mean a single processor or multiple processors, at the same or different locations, that cooperate to carry out the functionality described.

The memory 112 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface 114 may include a display device, speaker, microphone, touchscreen, mouse, keyboard, buttons, the like, or a combination thereof to allow for operator input and/or output.

Instructions 120 may be provided to carry out the functionality and methods described herein. The instructions 120 may be directly executed, such as a binary file, and/or may include interpretable code, bytecode, source code, or similar instructions that may undergo additional processing to be executed. The instructions 120 may be stored in the memory 112.

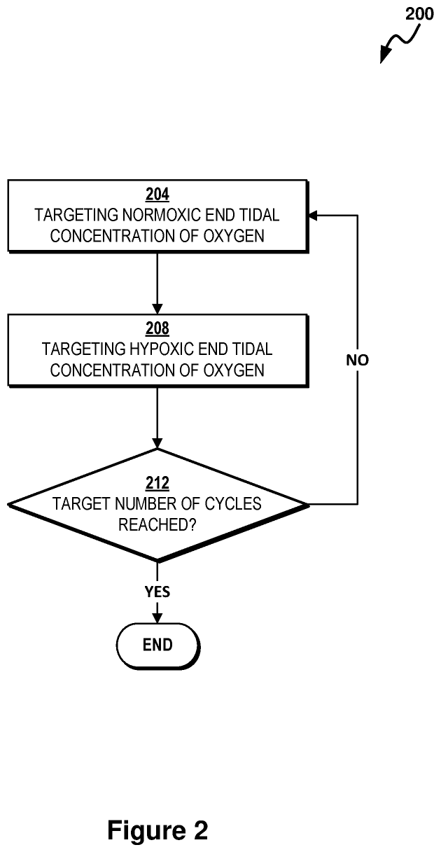

FIG. 2 is a flowchart showing an example method for providing intermittent hypoxia to a subject. The method 200 may be implemented by instructions 120 which control the system 100.

At block 204, the processor 110 controls the system 100 to induce a normoxic end tidal partial pressure of oxygen (P_{ET}O₂) in the subject 130.

Using the system 100, the normoxic P_{ET}O₂ may be reached rapidly. In specific examples, the normoxic P_{ET}O₂ is induced within a first number of breaths. In specific examples, the hypoxic P_{ET}O₂ is induced within one breath. In further examples, the hypoxic P_{ET}O₂ is induced within two breaths. In yet further examples, the hypoxic P_{ET}O₂ is induced within three breaths. If the subject's P_{ET}O₂ is hypoxic prior to the performance of block 204, the normoxic P_{ET}O₂ can be attained within one breath, or advantageously within 1 second.

The system 100 maintains the normoxic P_{ET}O₂ for a first duration of time. In specific non-limiting examples, the first duration of time is about 5 minutes. In specific non-limiting examples, the first duration of time is about 4 minutes. In other non-limiting examples, the first duration of time is about 3 minutes. In other non-limiting examples, the first duration of time is about 2 minutes. In other non-limiting examples, the first duration of time is about 1 minute.

The normoxic P_{ET}O₂ may be selected to induce a blood oxygen saturation that is considered normal for the human body. In particular examples, the normoxic P_{ET}O₂ is between about 70 mmHg and about 110 mmHg. In other examples, the normoxic P_{ET}O₂ is between about 95 mmHg and about 100 mmHg. In specific examples, the normoxic P_{ET}O₂ is about 95 mmHg.

At block 208, the processor 110 controls the system 100 to induce a hypoxic P_{ET}O₂ in the subject 130. Using the system 100, hypoxia may be attained rapidly. In some examples, the hypoxic P_{ET}O₂ may be induced within 1 minute, or advantageously within 30 seconds. In other examples, the hypoxic P_{ET}O₂ may be induced within a second number of breaths. In specific examples, the hypoxic P_{ET}O₂ is induced within one breath. In further examples, the hypoxic P_{ET}O₂ is induced within two breaths. In yet further examples, the hypoxic P_{ET}O₂ is induced within three breaths.

The system 100 maintains the hypoxic P_{ET}O₂ for a second duration of time. In specific non-limiting examples, the second duration of time is about 180 seconds. In a further non-limiting example, the second duration of time is about 120 seconds. In specific non-limiting examples, the second duration of time is about 90 seconds. In other non-limiting examples, the second duration of time is about 60 seconds. In other non-limiting examples, the second duration of time is about 40 seconds. In other non-limiting examples, the second duration of time is about 20 seconds.

The hypoxic P_{ET}O₂ may be selected to induce a blood oxygen saturation that is considered reduced for the human body. In particular examples, the hypoxic P_{ET}O₂ is between about 40 mmHg and about 80 mmHg. In specific non-limiting examples, the normoxic P_{ET}O₂ is about 40 mmHg. In other non-limiting examples, the normoxic P_{ET}O₂ is about 50 mmHg. In specific non-limiting examples, the normoxic P_{ET}O₂ is about 60 mmHg. In specific non-limiting examples, the normoxic P_{ET}O₂ is about 70 mmHg. In specific non-limiting examples, the normoxic P_{ET}O₂ is about 80 mmHg. Generally, a P_{ET}O₂ below 40 mmHg is considered harmful and should be avoided.

As will be understood by a person skilled in the art, blocks 204 and 208 can be performed in any order. In some examples, the system 100 induces a hypoxic P_{ET}O₂ and then induces a normoxic P_{ET}O₂. In other examples, the system 100 induces a normoxic P_{ET}O₂ and then induces a hypoxic P_{ET}O₂. In further examples, the system 100 targets a baseline P_{ET}O₂ before performing blocks 204 and 208.

The system 100 can target the normoxic and hypoxic end tidal concentrations of oxygen in the subject independently of the extent and pattern of the subject's breath

As part of blocks 204 and 208, the system 100 may control the end tidal partial pressure of carbon dioxide (P_{ET}CO₂) in the subject, independently of the P_{ET}O₂. In some examples, the system 100 induces a normocapnic P_{ET}CO₂ in the subject while varying the P_{ET}O₂. In other examples, the system 100 induces a hypercapnic P_{ET}CO₂ while varying the P_{ET}O₂. In yet examples, the system 100 induces a hypocapnic P_{ET}CO₂ while varying the P_{ET}O₂. In further examples, the system 100 induces a normoxic P_{ET}CO₂ while performing block 204 and a hypercapnic P_{ET}CO₂ while performing block 208. In further examples, the system 100 induces a hypocapnic P_{ET}CO₂ while performing block 204 and a normocapnic P_{ET}CO₂ while performing block 208. In further examples, the system 100 induces a hypercapnic P_{ET}CO₂ while performing block 204 and a normocapnic P_{ET}CO₂ while performing block 208. Inducing hypercapnia during the performance of method 200 enhances the therapeutic effects of intermittent hypoxia.

Generally, a normocapnic P_{ET}CO₂ is a partial pressure of CO₂ between about 30 mmHg and above 50 mmHg. Generally, a hypercapnic P_{ET}CO₂ is a partial pressure of CO₂ above 45 mmHg. Generally, a hypocapnic P_{ET}CO₂ is considered to be a partial pressure of CO₂ below 35 mmHg.

At block 212, the processor 110 determines whether the target number of cycles has been reached. Each repetition of blocks 204 to 208 is herein referred to as a cycle. Generally, the target number of cycles is selected to attain a therapeutically effective dose of intermittent hypoxia.

In specific non-limiting examples, the target number of cycles is two. In specific non-limiting examples, the target number of cycles is four. In specific non-limiting examples, the target number of cycles is six. In specific non-limiting examples, the target number of cycles is eight. In specific non-limiting examples, the target number of cycles is ten. In specific non-limiting examples, the target number of cycles is twelve. In specific non-limiting examples, the target number of cycles is fourteen. In specific non-limiting examples, the target number of cycles is eighteen. In specific non-limiting examples, the target number of cycles is twenty.

Upon performance of block 212, the target number of cycles may be retrieved from memory 112 or received as an input at the user interface 114. In some examples, the target number of cycles is selected according to the subject's demographics, the subject's identity, the disease, the therapeutic goal, the target number of sets per day, the target number of days, or a combination thereof.

If the processor 110 determines that the target number of cycles has not been reached, the method 200 returns to block 204 and repeats blocks 204 and 208 for another cycle.

If the processor 110 determines that the target number of cycles has been reached, the method 200 ends. As part of block 212, the processor 110 may control the user interface 114 to output a message indicating that the mask 108 can be removed. In some examples, if the processor 110 determines that the target number of cycles has been reached, the processor 110 controls the system 100 to target a normoxic P_{ET}O₂. As will be understood, target a normoxic P_{ET}O₂ after performance of method 200 may allow the subject 130 to breathe comfortably until the mask 108 is removed.

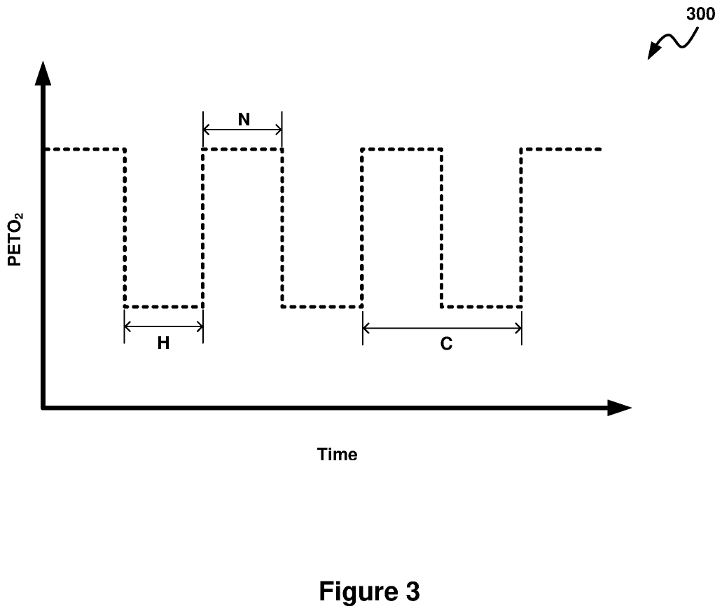

FIG. 3 is a graph showing exemplary performance of method 200. Time is indicated on the x axis, and P_{ET}O₂ is indicated on the y axis. N represents the first duration of time during which the system 100 induces a normoxic P_{ET}O₂, H represents the second duration of time during which the system 100 induces a hypoxic P_{ET}O₂, and C represents the cycle. In this example, the number of cycles C is three, since blocks 204 and 208 are performed three times, however the number of cycles C is not particularly limited.

In FIG. 3, the first duration N is approximately equal to the second duration H, however the first and second durations are not particularly limited. In other examples, the first duration N is longer than the second duration H. In yet other examples, the second duration H is longer than the first duration N.

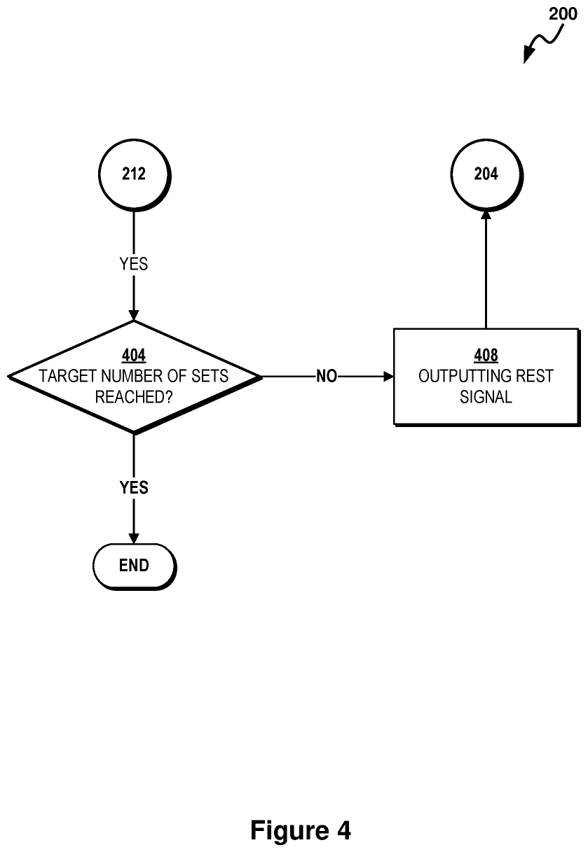

FIG. 4 shows another exemplary performance of method 200 in which blocks 204 to 212 are repeated.

After the processor 110 determines that the target number of cycles has been reached at block 212, the method proceeds to block 404. At block 404, the processor 110 determines whether the target number of sets has been reached. Each repetition of blocks 204 to 212 is herein referred to as a set. Generally, the target number of sets is selected to attain a therapeutically effective dose.

In specific non-limiting examples, the target number of sets is one. In specific non-limiting examples, the target number of sets is two. In specific non-limiting examples, the target number of sets is four. In specific non-limiting examples, the target number of sets is six. In specific non-limiting examples, the target number of sets is eight.

Upon performance of block 212, the target number of sets may be retrieved from memory 112 or received as an input at the user interface 114. In some examples, the target number of sets is selected according to the subject's demographics, the subject's identity, the disease, therapeutic goal, the number of prior sets, the target number of sets per day, the target number of days, the target number of subsequent sets, or a combination thereof.

If the processor 110 determines that the target number of sets has not been reached, the method 200 proceeds to block 408. At block 408, the processor 110 outputs a rest signal during a rest period.

In specific non-limiting examples, the rest period is about 5 minutes. In specific non-limiting examples, the rest period is about 4 minutes. In other non-limiting examples, the rest period is about 3 minutes. In other non-limiting examples, the rest period is about 2 minutes. In other non-limiting examples, the rest period is about 1 minutes. In other non-limiting examples, the rest period is about 30 seconds.

In response to receiving the rest signal, the user interface 114 may output a message indicating that the set is complete. The message may further communicate to the user that another set is required. The message may further communicate to the user the duration of the rest period, the rest period corresponding to the duration of time between the end of the previous set to the beginning of the next set. The message includes but is not limited to text, an image, a pre-recorded audio message, a light, or combinations thereof. In a specific non-limiting example, the rest signal controls the user interface 114 to display a countdown timer that displays the time until the rest period ends. In a further non-limiting example, the user interface 114 includes a speaker that plays a pre-recorded audio message indicating when the rest period has ended.

When the rest period ends, the method 200 returns to block 204. In some examples, the method 200 only returns to block 204 once the processor 110 receives a restart signal input at the user interface 114. During the rest period, the subject 130 may have removed the mask 108. The restart signal confirms to the processor 110 that the subject 130 is ready to begin another set.

If the processor 110 determines that the target number of sets has been reached, the method 200 ends. As part of block 404, the processor 110 may control the user interface 114 to output a message indicating that the treatment has ended. The message includes but is not limited to text, an image, a pre-recorded audio message, a light, or combinations thereof. In specific examples, the processor 110 controls the user interface 114 to display a message indicating that the mask 108 can be removed from the subject 130.

In some examples, if the processor 110 determines that the target number of sets has been reached, the processor 110 controls the system 100 to target a normoxic P_{ET}O₂. As will be understood, target a normoxic P_{ET}O₂ after performance of method 200 may allow the subject 130 to breathe comfortably until the mask 108 is removed from the subject 130.

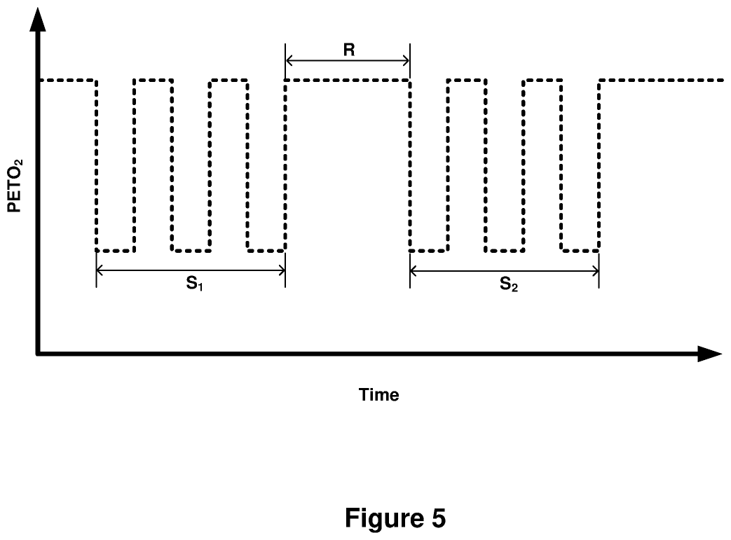

FIG. 5 is a graph depicting exemplary performance of method 200 in which blocks 204 to 212 are repeated. Time is indicated on the x axis, and P_{ET}O₂ is indicated on the y axis. R represents the rest period between a first set S₁ and a second set S₂. In this example, the system 100 induces normoxia during the rest period R, however the system 100 is not particularly limited. In other examples, the system 100 induces hyperoxia during the rest period R. In yet other examples, the subject 130 removes the mask 108 during the rest period R and the system 100 does not deliver gas to the subject 130 during the rest period R.

The method 200 may be repeated over the course of a treatment plan. In some non-limiting examples, the method 200 is repeated two or more times in one day. In non-limiting examples, the method 200 is repeated on a daily, weekly, biweekly, monthly, or bimonthly basis. The method 200 may be repeated until a desired physiological outcome is attained.

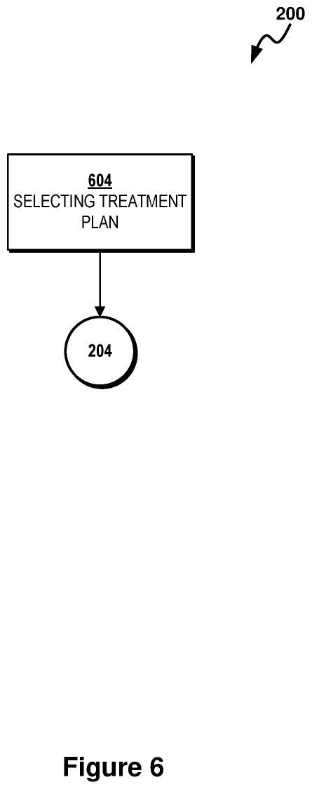

Before inducing intermittent hypoxia in the subject, a treatment may be selected, as shown at FIG. 6. Block 604 comprises selecting a treatment plan and is performed before block 204 in method 200. The treatment plan comprises parameters for providing the subject with a dose of intermittent hypoxia. The parameters include, but are not limited to, the first duration, the normoxic P_{ET}O₂, the second duration, the hypoxic P_{ET}O₂, the first P_{ET}CO₂, the second P_{ET}CO₂, the target number of cycles, the target number of sets, the rest period R, the target number of sets per day, the target number of days, or combinations thereof.

In some examples, block 604 comprises receiving an input at the user interface 114, the input comprising the parameters for the treatment plan. In response to receiving the parameters, the processor 110 controls the system 100 to perform method 200 according to the parameters input at the user interface 114.

In other examples, block 604 comprises retrieving a treatment plan from the memory 112. In these examples, the memory 112 stores a database comprising one or more treatment plans. The one or more treatment plans comprise parameters for delivering a dose of intermittent hypoxia. The parameters are stored in memory 112 in association with one or more reference attributes. Reference attributes include, but are not limited to, subject identity, age, sex, race, body weight, height, lung capacity, health conditions, reason for treatment, and combinations thereof. As part of block 604, the user interface 114 receives inputs indicating one or more attributes of the subject 130. In response to receiving the inputs, the processor 110 retrieves a treatment plan from memory 112, the retrieved treatment plan selected based on a comparison between the subject attributes and reference attributes stored in the database. Method 200 is then performed according to the parameters of the retrieved treatment plan in order to provide the subject 130 with a dose of intermittent hypoxia.

There are numerous practical applications for the system 100 and method 200.

The system 100 can be used in the treatment of lung disorders including but not limited to chronic obstructive lung disease, emphysema, bronchitis, and asthma. Applying intermittent hypoxia using the system 100 can improve ventilatory and cardiovascular function in patients with lung disorders.

The system 100 can be used in the treatment of spinal cord injuries. Applying intermittent hypoxia using the system 100 can improve motor and respiratory function following a spinal cord injury.

The system 100 can be used to improve cognitive function in the elderly. The system 100 can be used to improve cognitive function in subjects with Alzheimer's disease or dementia.

The system 100 can be used in the treatment of depression.

The system 100 can be used in the treatment of cardiovascular disorders including, but not limited to, myocardial ischemia, angina, myocardial infarction, coronary artery disease, and heart failure. Applying intermittent hypoxia using the system 100 can improve physical performance and quality of life in subjects with cardiovascular disorders.

The system 100 can be used in the treatment of hypertension.

The system 100 can be used to treat metabolic syndrome. The system 100 can be used to normalize abnormal lipid metabolism.

The system 100 can be used in the treatment of chronic systemic inflammation.

The system 100 can be used to precondition a tissue or organ for surgery.

The system 100 can be used to precondition a tissue or organ for surgery, in particular a surgery that is likely to interrupt the blood supply to the tissue or organ. In some examples, the surgery is an organ or tissue transplantation. During transplantation, the organ or tissue is deprived of oxygen for the period of time that it takes to remove the organ or tissue from the donor and reattach the organ or tissue to blood vessels in the recipient. As a result, the organ or tissue can experience oxidative stress, ischemia, and reperfusion syndrome. Although chilling the organ or tissue can extend the life of the organ or tissue, ice crystals cause cellular damage. The system 100 described herein can be used to improve outcomes for transplantation. By performing the method 200 on the donor prior to the transplantation, the organ or tissue develops resistance to hypoxia. This can extend the lifetime of the organ or tissue and improve outcomes for the recipient.

The system 100 can be used to increase aerobic capacity in a subject. In particular non-limiting examples, the system 100 is used to increase aerobic capacity in elderly males. In particular non-limiting examples, the system 100 is used to increase aerobic capacity in athletes.

The system 100 can be used to stimulate erythropoietin production in a subject.

The system 100 can be used to control metabolism syndrome. In particular, the system 100 can be used to reduce body weight, lower cholesterol, normalize blood sugar levels, and increase insulin sensitivity.

The system 100 can be used to improve bone remodeling. The system 100 can be used to increase osteoblast formation. The system 100 can be used to suppress osteoclast formulation.

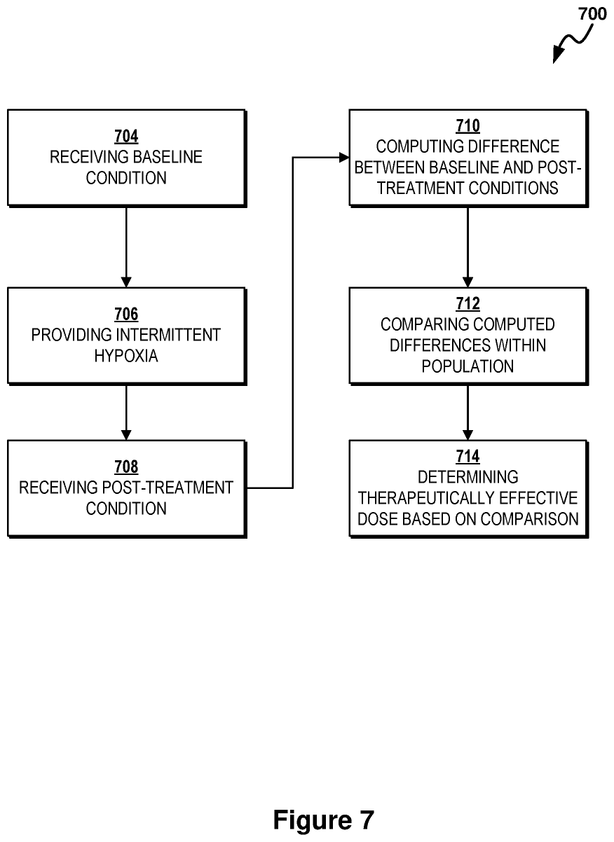

FIG. 7 is a flowchart depicting a method 700 of determining a therapeutically effective dose of intermittent hypoxia. In FIG. 7, the method 700 is performed by system 100.

At block 704, the system 100 receives a baseline condition for a population of subjects. As part of block 704, the system 100 may receive the baseline condition as an input received at the user interface 114. The baseline condition includes, but is not limited to, cardiovascular function, respiratory function, cognitive function, memory, reflexes, coordination, strength, mood, cerebral spinal fluid (CSF) composition, blood composition, body weight, exercise tolerance, altitude tolerance, aerobic capacity, athletic endurance, or combinations thereof. Generally, the system 100 receives the baseline condition for each subject in the population of subjects.

At block 706, the system 100 provides intermittent hypoxia to the population of subjects. The intermittent hypoxia may be provided according to the method 200.

In some examples, the population of subjects includes at least one test group and at least one control group. The test group is provided with a test dose of intermittent hypoxia and the control group is provided with a control dose of intermittent hypoxia that is different from the test dose. The test and control doses of intermittent hypoxia include, but are not limited to, a partial pressure of end tidal oxygen induced during normoxia, a partial pressure of end tidal oxygen induced during hypoxia, a partial pressure of a carbon dioxide induced during normoxia, a partial pressure of a carbon dioxide induced during hypoxia, a first duration, a second duration, a target number of cycles, a rest period, a total number of sets, a target number of sets per day, a target number of days, and combinations thereof. In some examples, the control group is not provided with intermittent hypoxia.

It should be understood by a person skilled in the art that the population of subjects may include multiple test groups and multiple control groups in order to evaluate the efficacy of multiple doses of intermittent hypoxia.

In some examples, the test group and the control group are selected to share a common attribute. The common attribute includes, but is not limited to, health condition, pathological condition, sex, age, race, body weight, height, or combinations thereof. In examples where the common attribute is a pathological condition, the common attribute includes, but is not limited to, chronic obstructive lung disease, emphysema, bronchitis, asthma, spinal cord injury, Alzheimer's disease, dementia, depression, myocardial ischemia, angina, myocardial infarction, coronary artery disease, heart failure, hypertension, metabolic syndrome, inflammatory disease, lung disease, or combinations thereof. In examples where the common attribute is a health condition, the common attribute includes, but is not limited to, exercise tolerance, altitude tolerance, aerobic capacity, and athletic endurance.

At block 708, the system 100 receives a post-treatment condition for the population of subjects. As part of block 708, the system 100 may receive the post-treatment condition as an input received at the user interface 114. The post-treatment condition includes, but is not limited to, cardiovascular function, respiratory function, cognitive function, memory, reflexes, coordination, strength, mood, cerebral spinal fluid (CSF) composition, blood composition, body weight, exercise tolerance, altitude tolerance, aerobic capacity, athletic endurance, or combinations thereof. For comparison purposes, the post-treatment condition typically corresponds with the baseline condition. Generally, the system 100 receives the post-treatment condition for each subject in the population of subjects.

At block 710, the processor 110 computes the difference between the baseline condition and the post-treatment condition for the population of subjects. The difference between the baseline condition and the post-treatment condition may be indicative of the efficacy of the intermittent hypoxia on the respective individual. In some examples, the difference between the baseline and post-treatment conditions is a positive value, indicating that the condition of the subject improved after receiving a dose of intermittent hypoxia. In other examples, the difference between the baseline and post-treatment conditions is a negative value, indicating that the condition of the subject declined after receiving the treatment. In further examples, the difference between the baseline and post-treatment conditions is zero or negligible, indicating that the dose had no effect on the subject.

At block 712, the processor 110 compares the computed differences within the population. In examples where the population includes a test group and a control group, block 712 comprises comparing the computed differences for the test group to the computed differences for the control group. The comparison may include any suitable statistical method known in the art.

At block 714, the processor 110 determines a therapeutically effective dose of intermittent hypoxia based on the comparison at block 712. If the condition of the subjects in the test group improved more than the condition of the subjects in the control group, the test dose of intermittent hypoxia is therapeutically effective.

While method 700 has been described with respect to a population of subjects, it should be understood that a variation on method 700 may instead be used to determine a therapeutically effective dose for an individual. In this variation, a condition of the individual is assessed before and after a first dose of intermittent hypoxia and before and after a second dose of intermittent hypoxia. The processor 110 compares the change in condition resulting from the first dose with the change in condition resulting from the second dose to determine whether the first or second dose of intermittent hypoxia is therapeutically effective.

It will now be apparent to a person of skill in the art that the present specification affords certain advantages over the prior art. Current practices of providing intermittent hypoxia to a subject use a non-breathing mask to deliver a controlled concentration of oxygen to a subject. These devices rely on progressive trial and error to target and maintain P_{ET}O₂ which prolongs the cycle time and consumption of source gas. Furthermore, targeting PₐO₂ with fixed inspired gas precludes the coordinated control of PₐCO₂ which varies with minute ventilation and may act as a confounder. Significantly, the PₐO₂ is unknown, and repeatability is not guaranteed.

In contrast, the system 100 and method 200 targets the P_{ET}O₂ quickly and precisely using sequential gas delivery. This improves subject tolerance of the treatment, reduces use of the source gas, and ensures that the subject receives a therapeutic dose without reaching damaging levels of hypoxia. Furthermore, the system 100 can control the P_{ET}CO₂ independently of the P_{ET}O₂, which intensifies the effects of intermittent hypoxia. Importantly, the method 200 is repeatable, ensuring consistency between treatments and allowing providers for the first time to determine dosage effects of hypoxia. Using the method 200 and system 100, it is possible to ascertain a therapeutic dose for an individual or a population.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A method of treating a pathological condition in a subject, the method comprising:
(a) inducing a normoxic end tidal concentration of oxygen in the subject within a first number of breaths, using a sequential gas delivery system and maintaining the normoxic end tidal concentration of oxygen in the subject for a first duration of time;
(b) inducing a hypoxic end tidal concentration of oxygen in the subject within a second number of breaths using the sequential gas delivery system and maintaining the hypoxic end tidal concentration of oxygen in the subject for a second duration of time; and
(c) repeating (a) and (b) for a target number of cycles to attain a therapeutically effective dose.

2. The method of claim 1 wherein the first number of breaths is one.

3. The method of claim 1 wherein the second number of breaths is one.

4. The method of claim 1 wherein the normoxic and hypoxic end tidal concentrations of oxygen in the subject are controlled independently of the extent and pattern of the subject's breath.

5. The method of claim 1 further comprising the step of maintaining an end tidal concentration of carbon dioxide in the subject using the sequential gas delivery system while performing (a) to (c).

6. The method of claim 1 further comprising:
inducing a first end tidal concentration of carbon dioxide in the subject using the sequential gas delivery system and maintaining the first end tidal concentration of carbon dioxide while inducing the normoxic end tidal concentration of oxygen; and
inducing a second end tidal concentration of carbon dioxide in the subject using the sequential gas delivery system and maintaining the second end tidal concentration of carbon dioxide while inducing the hypoxic end tidal concentration of oxygen.

7. The method of claim 6 wherein the first end tidal concentration of carbon dioxide is selected to induce normocapnia and the second end tidal concentration of carbon dioxide is selected to induce hypercapnia.

8. The method of claim 1 further comprising:
inducing a normoxic end tidal concentration of oxygen and a normocapnic end tidal concentration of carbon dioxide in the subject for a rest period; and
repeating (a) to (c) after the rest period.

9. The method of claim 8 wherein performance of (a) to (c) constitutes a set, the method further comprising the step of repeating the set for a pre-determined number of sets.

10. The method of claim 9 further comprising the step of repeating a set for a predetermined number of times in a day.

11. The method of claim 9 or 10 further comprising the step of repeating a set for a pre-determined number of days.

12. The method of any one of claims 1 to 11 wherein the pathological condition is one of chronic obstructive lung disease, emphysema, bronchitis, asthma, spinal cord injury, Alzheimer's disease, dementia, depression, myocardial ischemia, angina, myocardial infarction, coronary artery disease, heart failure, hypertension, metabolic syndrome, inflammatory disease, and lung disease.

13. A method of improving a health condition of a subject, the method comprising:
(a) inducing a normoxic end tidal concentration of oxygen within a first number of breaths using a sequential gas delivery system and maintaining the normoxic end tidal concentration of oxygen for a first duration of time;
(b) inducing a hypoxic end tidal concentration of oxygen within a second number of breaths using the sequential gas delivery system and maintaining the hypoxic end tidal concentration of oxygen for a second duration of time; and
(c) repeating (a) and (b) for a target number of cycles to attain a therapeutically effective dose.

14. The method of claim 13 wherein the first number of breaths is one.

15. The method of claim 13 wherein the second number of breaths is one.

16. The method of claim 13 wherein the normoxic and hypoxic end tidal concentrations of oxygen in the subject are controlled independently of the extent and pattern of the subject's breath.

17. The method of claim 13 further comprising the step of maintaining an end tidal concentration of carbon dioxide in the subject using the sequential gas delivery system while performing (a) to (c).

18. The method of claim 13 further comprising:
inducing a first end tidal concentration of carbon dioxide in the subject using the sequential gas delivery system and maintaining the first end tidal concentration of carbon dioxide while inducing the normoxic end tidal concentration of oxygen; and
inducing a second end tidal concentration of carbon dioxide in the subject using the sequential gas delivery system and maintaining the second end tidal concentration of carbon dioxide while inducing the hypoxic end tidal concentration of oxygen.

19. The method of claim 18 wherein the first end tidal concentration of carbon dioxide is selected to induce normocapnia and the second end tidal concentration of carbon dioxide is selected to induce hypercapnia.

20. The method of claim 13 further comprising the step of inducing a normoxic end tidal concentration of oxygen and a normocapnic end tidal concentration of carbon dioxide for a rest period and repeating (a) to (c) after the rest period.

21. The method of claim 20 wherein performance of (a) to (c) comprises a set, the method further comprising the step of repeating the set for a pre-determined number of sets.

22. The method of claim 21 further comprising the step of repeating the set for a predetermined number of times in a day.

23. The method of claim 21 or 22 further comprising the step of repeating the set for a predetermined number of days.

24. The method of claim 13 wherein the health condition is one of exercise tolerance, altitude tolerance, aerobic capacity, and athletic endurance.

25. Use of a sequential gas delivery system in the treatment of a pathological condition.

26. The use according to claim 25 wherein the pathological condition is one of chronic obstructive lung disease, emphysema, bronchitis, asthma, spinal cord injury, Alzheimer's disease, dementia, depression, myocardial ischemia, angina, myocardial infarction, coronary artery disease, heart failure, hypertension, metabolic syndrome, inflammatory disease, and lung disease.

27. Use of a sequential gas delivery system in the enhancement of a health condition.

28. The use according to claim 27 wherein the health condition is one of exercise tolerance, altitude tolerance, aerobic capacity, and athletic endurance.

29. Use of a sequential gas delivery system to precondition an organ or tissue prior to a surgery.

30. The use of claim 29 wherein the surgery is a transplantation of the organ or tissue.

31. Use of a sequential gas delivery system to improve bone remodelling.

32. Use of a sequential gas delivery system to stimulate erythropoietin production in a subject.

33. A method of determining a therapeutically effective dose of hypoxia, the method comprising:
(a) assessing a baseline condition for a population of subjects;
(b) providing intermittent hypoxia to the population of subjects using a sequential gas delivery system;
(d) assessing a post-treatment condition for the population of subjects;
(e) computing the difference between the baseline condition and the post-treatment condition for the population of subjects;
(f) comparing the computed differences within the population of subjects; and
(g) determining a therapeutically effective dose of intermittent hypoxia based on the comparison.

34. The method of claim 33 wherein the population of test subjects includes a test group and a control group, and wherein providing intermittent hypoxia to the population of subjects comprises:
providing a test dose of intermittent hypoxia to the test group; and
providing a control dose of intermittent hypoxia to the control group.

35. The method of claim 34 the test and control doses of intermittent hypoxia are characterized by at least one of a partial pressure of end tidal oxygen induced during normoxia, a partial pressure of end tidal oxygen induced during hypoxia, a partial pressure of a carbon dioxide induced during normoxia, a partial pressure of a carbon dioxide induced during hypoxia, a first duration, a second duration, a target number of cycles, a rest period, a total number of sets, a target number of sets per day, and a target number of days.

36. The method of claim 35 wherein comparing the computed differences within the population of subjects comprises the step of comparing the computed differences for the test group to the computed differences for the control group.

37. The method of claim 33 wherein the population of subjects is selected to share a common attribute.

38. The method of claim 37 wherein the common attribute is one of a health condition, pathological condition, sex, age, race, body weight, and height.

39. The method of claim 37 wherein the common attribute is one of chronic obstructive lung disease, emphysema, bronchitis, asthma, spinal cord injury, Alzheimer's disease, dementia, depression, myocardial ischemia, angina, myocardial infarction, coronary artery disease, heart failure, hypertension, metabolic syndrome, inflammatory disease, and lung disease.

40. The method of claim 37 wherein the common attribute is one of exercise tolerance, altitude tolerance, aerobic capacity, and athletic endurance.
